(19) United States
(12) Patent Application Publication
(10) Pub. No.: US 2010/0094163 A1
(43) Pub. Date: Apr. 15, 2010

(54) MEDICAL APPARATUS WITH A SENSOR FOR DETECTING A FORCE

(75) Inventors: Szabolcs Deladi, Eindhoven (NL); Nenad Mihajlovic, Eindhoven (NL); Joachim Kahlert, Aachen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/529,372

(22) PCT Filed: Mar. 4, 2008
(86) PCT No.: PCT/IB08/50781

§ 371 Date: Sep. 1, 2009

(30) Foreign Application Priority Data
Mar. 7, 2007 (EP) 07103658.6

(51) Int. Cl. A61B 5/00; H05K 13/00

(52) U.S. Cl. 600/561; 29/592.1

(57) ABSTRACT

The invention relates to a medical apparatus (120,130) having a sensor (100,110) for detecting a force acting on the medical apparatus (120,130) in a longitudinal direction. The medical apparatus (120,130) comprises an opto-mechanical force transducer having a flexible part (22,42) for receiving the force, an optical guide (1) having an outcoupling surface (11) that faces the flexible part (22,42) of the opto-mechanical force transducer, and a photodetector which detects an interference pattern composed of light (32) in the optical guide (1) that is reflected from the outcoupling surface (11) of the optical guide (1) and of light (33) in the optical guide (1) that is reflected from the flexible part (11) of the opto-mechanical force transducer. The use of the interference pattern composed of the light reflected from the out-coupling surface of the optical guide and the light reflected from the flexible part of the opto-mechanical force transducer results in a more accurate measurement of the force acting on the medical apparatus.

### FIELD OF THE INVENTION

The invention relates to a medical apparatus for detecting a force that acts on the medical apparatus in the longitudinal direction.

### BACKGROUND OF THE INVENTION

Such a sensor is known from WO 2005/011511 A1 which discloses a sensor for detecting a force acting on an elongate device, especially an elongate device such as a catheter, said force comprising a non-neglectable force component in the longitudinal direction of the elongate device. The sensor encompasses a force transducer for the force that is to be detected, a connection for mounting the sensor on the elongate device, at least one light input area which can be optically connected to at least one optical waveguide that injects light into the sensor, a light intensity modulator which modulates a predetermined intensity of light that can be injected into the sensor according to the force applied to the force transducer, and at least one light-decoupling area via which the light having modulated intensity can be decoupled in at least one optical guide. A disadvantage of the known sensor is the relatively low accuracy of the detected force because of the modulated light intensity.

### SUMMARY OF THE INVENTION

It is an object of the invention to provide a medical apparatus having a sensor for detecting a force that acts on the medical apparatus in the longitudinal direction with an improved accuracy. The invention is defined by the independent claims. Advantageous embodiments are defined by the dependent claims.

This object is achieved by the medical apparatus according to the invention, which is characterized in that the medical apparatus comprises an opto-mechanical force transducer having a flexible part for receiving the force, an optical guide having an outcoupling surface that faces the flexible part of the opto-mechanical force transducer, and a photodetector which detects an interference pattern composed of light in the optical guide that is reflected from the outcoupling surface of the optical guide and of light in the optical guide that is reflected from the flexible part of the opto-mechanical force transducer. The use of the interference pattern composed of the light reflected from the outcoupling surface of the optical guide and the light reflected from the flexible part of the opto-mechanical force transducer results in a more accurate measurement of the force acting on the medical apparatus, because the wavelength of light is the basis for the measurement instead of the intensity of light, which wavelength is a parameter that results in more accurate measurement results than the intensity of light.

WO 2006/092707 A1 discloses an apparatus for diagnosing or treating an organ or vessel, wherein a deformable body having at least two optical fiber sensors disposed in a distal extremity thereof is coupled to processing logic programmed to compute a multi-dimensional force vector responsive to detected changes in the optical characteristics of the optical fiber sensors arising from deflection of the distal extremity resulting from contact with the tissue of the wall of the organ or vessel. The force vector may be used to facilitate manipulation of the deformable body either directly or automatically using a robotic system. The force measurement of this apparatus is less accurate than that in the medical apparatus according to the invention because a torque around the longitudinal axis of the medical apparatus causes a disturbance of the measurement of the force in the axial direction. Furthermore, the forces are not measured directly by the optical fiber sensors, instead a deformation of the deformable body is transferred to the optical fiber sensors, wherein the sensing part of the optical fiber sensors, such as for example a Bragg grating, is disposed within the deformable body and not within the distal extremity of the medical apparatus.

In an embodiment of the medical apparatus according to the invention, the flexible part of the opto-mechanical force transducer comprises a protrusion that receives the force. The protrusion advantageously provides for a well defined area on which the force acts that has to be measured. Furthermore, the protrusion avoids that the force to be measured acts on a part of the medical apparatus outside the force transducer resulting in an erroneous force measurement and detection.

In a further embodiment of the medical apparatus according to the invention, the force comprises a contact force between the protrusion and a tissue surface inside a bodily lumen. In this way the contact force of the medical apparatus with the tissue surface is available for the person that operates the medical apparatus. The knowledge of the contact force assists this person to avoid accidental damaging of the tissue due to excess force. Alternatively, in the case that the medical apparatus is operated automatically, for example by a robotic system, the measured force can be used by the automated system to control real-time the movement of the medical apparatus such that damaging of the tissue is avoided.

In another further embodiment of the medical apparatus according to the invention, the protrusion is located outside a path of light that is emitted from the outcoupling surface of the optical guide. This reduces any disturbing influence of the protrusion on the force measurement.

In another embodiment of the medical apparatus according to the invention, the medical apparatus comprises a closed space region at least confined by the flexible part and the outcoupling surface of the optical guide for detecting a pressure difference between the pressure of the environment and the pressure inside the closed space region. For example, the blood pressure inside the heart or the vessels of a bodily lumen can be measured using the medical apparatus according to this embodiment in which the pressure of the environment is the blood pressure inside the bodily lumen. Additionally, it is also possible to detect a contact force between the protrusion and a tissue surface inside a bodily lumen using this embodiment.

In an embodiment of the medical apparatus according to the invention, the flexible part of the opto-mechanical force transducer comprises a bottom surface facing the outcoupling surface of the optical guide and a top surface which receives the force and which is opposite to the bottom surface, and in which the flexible part is supported at its outer side by a rigid supporting part mounted on a part of the optical guide. This provides for a simple construction of the force transducer with the flexible part mounted on the optical guide via the rigid supporting part resulting in a well-defined relative position of the flexible part in relation to the outcoupling surface of the optical guide.

In a further embodiment of the medical apparatus according to the invention, the flexible part of the opto-mechanical force transducer comprises a flexible bridge connection supported on each side by a rigid bridge support mounted on a part of the optical guide in which the protrusion for receiving the force is located on the top surface of the flexible bridge connection. This embodiment enables an accurate measurement of the force that acts locally on the protrusion which is transferred into a bending of the flexible bridge connection. The rigid bridge support on each side of the flexible bridge connection advantageously provides for a solid mounting of the force transducer on the optical guide.

In another embodiment of the medical apparatus according to the invention, the flexible part of the opto-mechanical force transducer comprises a fiber material at least partly coated with a reflective material on the bottom surface or on the top surface of the flexible part. This embodiment enables a simplified manufacturing of the medical apparatus by using, for example, the same material for the optical fiber and the force transducer. By adding a reflective coating on one of the surfaces of the flexible part of the opto-mechanical force transducer, the intensity of the reflected light is increased resulting in a more accurate measurement of the interference pattern. Examples of reflective material include Pt and Au.

In an embodiment of the medical apparatus according to the invention, at least three opto-mechanical force transducers are mounted around a central elongate part. This advantageously provides for a determination of the spatial orientation of the force that acts on the medical apparatus. If the force that acts on the medical apparatus comprises a contact force between the medical apparatus and a tissue surface, the spatial orientation of the measured contact force enables the determination of the relative position of the medical apparatus with respect to the tissue surface. In this way the orientation and hence the angle can be determined that the medical apparatus has with respect to the tissue surface. For example, by keeping the contact forces on each of the force transducers considerably equal, the medical apparatus will be positioned essentially perpendicular to the tissue surface. In case an automated robotic system is applied for operation of the medical apparatus, the movement of the medical apparatus will be controlled by the robotic system in such a way that the medical apparatus is positioned in a required position with relation to the tissue surface based upon the measured contact forces of each of the three force transducers.

In an embodiment of the medical apparatus according to the invention, the medical apparatus further comprises processing logic for computing the force from the interference pattern. The processing logic can also be present outside the medical apparatus and a connection between the medical apparatus and any external apparatus can be implemented via a wireless connection in which the medical apparatus is adapted to be able to wirelessly connect to any external apparatus.

### BRIEF DESCRIPTION OF THE DRAWINGS

These and other aspects of the invention will be further elucidated and described with reference to the drawings, in which:

FIG. 1 is perspective view of an end part of a force sensor according to an embodiment of the invention;

FIGS. 2 and 3 are cross-sectional views of an end part of a force sensor according to an embodiment of the invention;

FIG. 4a is a perspective view and FIG. 4b a perspective cross-sectional view of an embodiment of an end part of a medical apparatus according to the invention;

FIGS. 5, 6 and 7 are perspective views of further embodiments of an end part of a medical apparatus according to the invention; and

FIGS. 8a-g show schematic perspective views of a method of manufacturing a force sensor according to an embodiment of the invention.

The Figures are not drawn to scale. In general, identical components are denoted by the same reference numerals in the Figures.

### DETAILED DESCRIPTION OF EMBODIMENTS

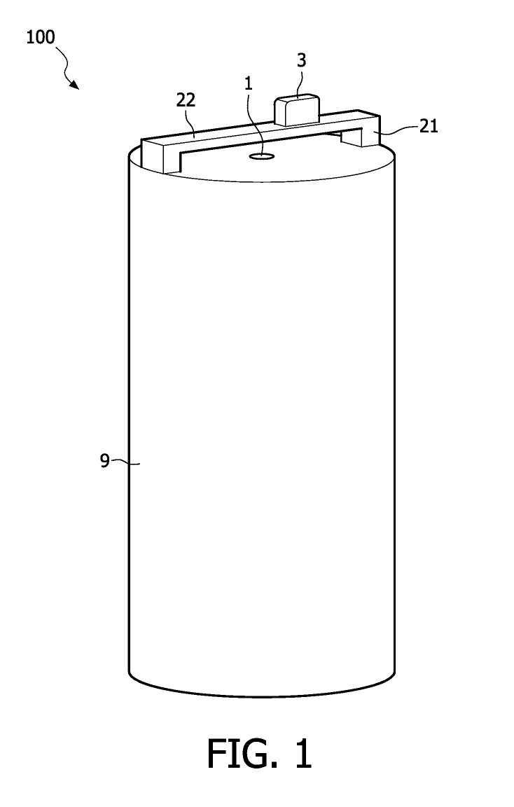

FIG. 1 shows a perspective view of an embodiment of an end part of a force sensor 100 according to an embodiment of the invention. The force sensor 100 is, for example, part of a catheter or an endoscope, or any apparatus used for diagnosis or treatment inside a bodily lumen. The end part of the force sensor 100 as shown in FIG. 1 comprises an elongated body 9, such as, for example, a cylinder, having an optical waveguide 1 in the middle for guiding light 30 originating from a laser source (not shown). The optical waveguide can, for example, be fabricated from a fiber material and can, for example, be surrounded by a material with a refractive index that is different from that of the optical waveguide 1. The force sensor 100 further comprises a sensor, in this case shaped as a bridge-like structure with a flexible part 22 supported on each side by rigid bridge supports 21. A protrusion 3 is mounted on a top surface 24 of the flexible part 22 for receiving a contact force F.

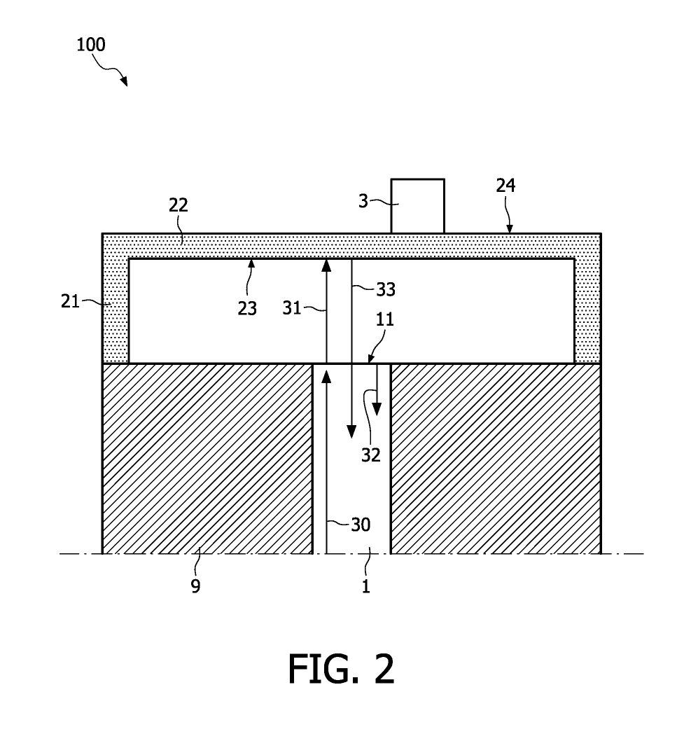

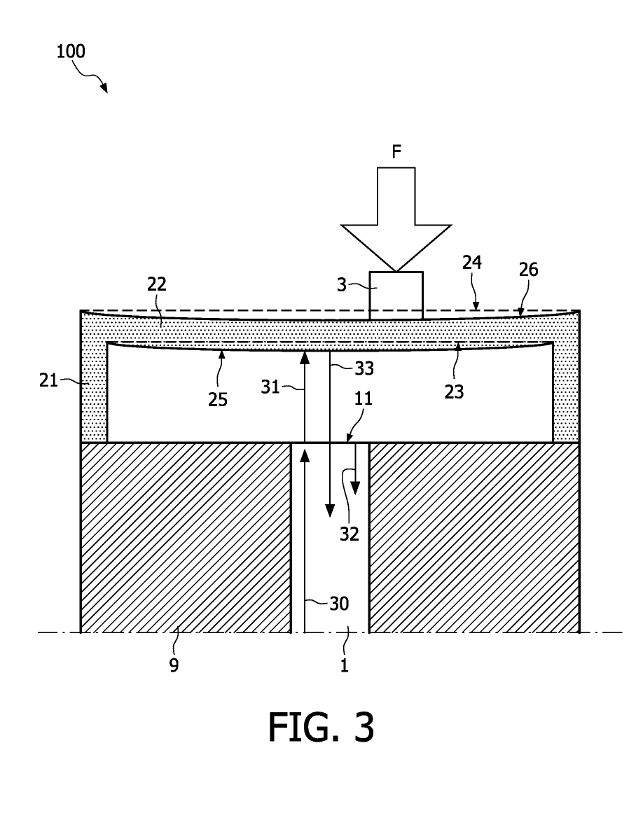

FIGS. 2 and 3 are cross-sectional views of the end part of the force sensor 100 according to an embodiment of the invention and further show the operation of the force sensor 100. FIG. 2 shows that a first part 31 the light 30 in the optical waveguide 1 that is originating from the laser source (not shown) exits from the optical waveguide 1 at an outcoupling surface 11. First reflected light 32 comprises a second part of the light 30 that is reflected at the outcoupling surface 11 and is guided in the opposite direction of the light 30 in the optical waveguide 1. Second reflected light 33 comprises a part of the first part 31 of the light 30 that is reflected at a bottom surface 23 of the flexible part 22 and that enters the optical waveguide 1 through the outcoupling surface 11. Hence, the optical waveguide 1 guides two light beams in a direction opposite to the direction of the light 30: the first reflected light 32, that is reflected from the outcoupling surface 11 and the second reflected light 33, that is reflected from the bottom surface 23 of the flexible part 22 and that entered the optical waveguide 1 through the outcoupling surface 11. These two reflected light beams 32 and 33 are combined resulting in an interference signal that is, for example, detected and measured at an end of the optical waveguide 1 that is opposite to the outcoupling surface 11 of the force sensor 100 via, for example, a photodetector that will produce a voltage or current signal.

FIG. 3 shows the force sensor 100 when a contact force F acts on the protrusion 3. The contact force F results from the protrusion 3 being in contact with a contact surface such as, for example, a soft tissue or the artery wall. The component of the contact force F, that is in the longitudinal direction or a direction perpendicular to the force sensor 100, results in a bending of the flexible part 22, resulting in a displacement of the bottom surface 23 to the deformed bottom surface 25 and of the top surface 24 to the deformed top surface 26. As a result of the displacement of the flexible part 22, the distance between the outcoupling surface 11 and the flexible part 22 is changed from the bottom surface 23 to the deformed bottom surface 25. Hence the length of the path of the second reflected light 33 is changed, which results in a change of the interference signal of the first reflected light 32 and the second reflected light 33. This change of the interference signal is then measured with, for example, the photodetector, and converted into a value for the displacement. Because the characteristics, such as stiffness, of the flexible part 22 are known the value of the contact force F can be computed from the measured and computed displacement. The characteristics of the flexible part 22 are designed such that the contact force F can be measured. For example, if the contact force F is expected to be between 0.2 Newton and 1.0 Newton, the flexible part will have a length of 40 micrometers, a width of 25 micrometers and a thickness of 20 micrometers. An embodiment of the flexible part 22 comprises, for example, silicon oxide, wherein the top surface 24 of the flexible part 22 is coated with a material that reflects light. Such a material is for example Pt or Au, and is at least applied on an area of the top surface 24 that is located above and facing the outcoupling surface 11. In this case, a part of the first part 31 of the light 30 will enter the flexible part 22 and will subsequently reflect from the top surface 24 and enter the optical waveguide 1. Furthermore, in this case the second reflected light 33 is only a fraction of the part of the first part 31 of the light 30 that enters the flexible part 22, for example only 4% reflects at the bottom surface 23 of the flexible part 22. Alternatively, a part of the bottom surface 23 of the flexible part 22 may be coated with the reflective material.

The shape of the protrusion 3 is such that it is able to receive the contact force F and that the corresponding load is transferred locally on the contact surface, but does not damage, for example cut, the contact surface which the protrusion 3 contacts. The form of the protrusion 3 is for example rectangular shaped for contact with soft tissue or pyramidal shaped for contact with a firm and solid target sample to transfer the load locally to the tissue. An alternative that can be sued in both cases is the use of a hemispherical shape for the protrusion 3. The protrusion 3 is in this example not located on a part of the top surface 24 that faces the outcoupling surface 11, which reduces any disturbing effect of the protrusion on the measured interference signal.

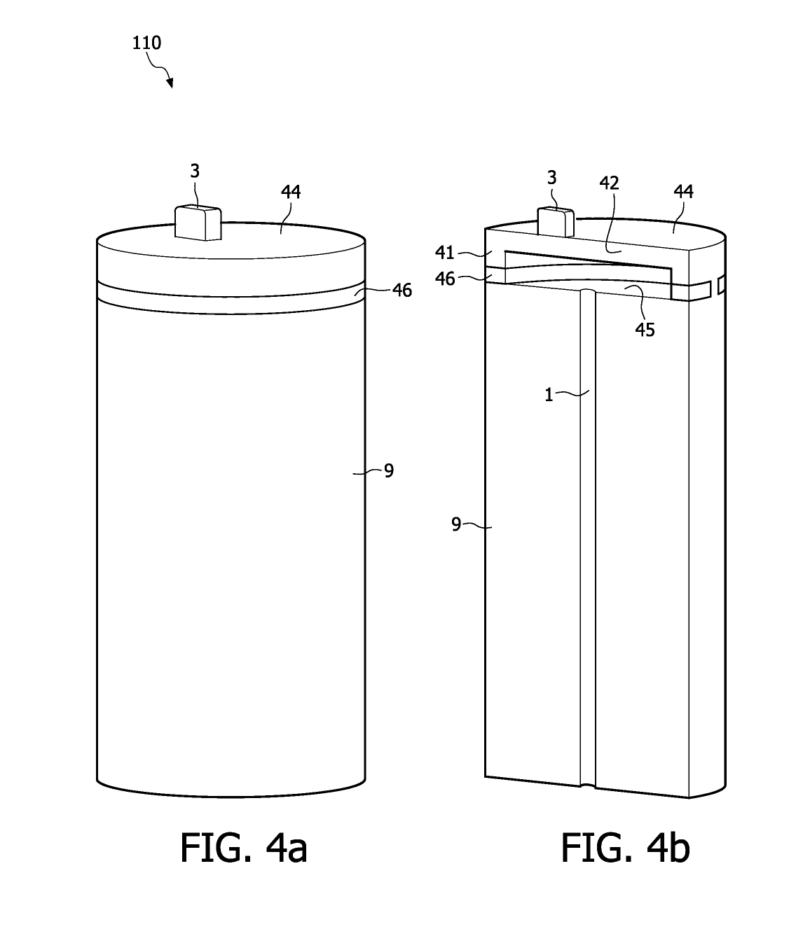

FIG. 4a shows a perspective view and FIG. 4b a perspective cross-sectional view of a force sensor 110 according to another embodiment of the invention. In this case the flexible part of the force sensor 110 is a flexible disc 42 having a top surface 44 on which the protrusion 3 is located. The flexible disc 42 is supported and mounted via an auxiliary material, such as, for example, a glue layer 46, on the elongated body 9 by a rigid ring support 41. The cylindrical body 9, the flexible disc 42 and the rigid ring support 41 enclose a sealed cavity 45. This enables measuring a pressure difference between the pressure inside the sealed cavity 45 and the pressure outside the sealed cavity 45. For example, the blood pressure in the heart or inside the vessel can be measured with this embodiment, in addition to the measurement of the contact force F acting on the protrusion 3. Also an embodiment without the protrusion 3 is possible in which then only this pressure difference is measured.

The flexible disc 42 can be fabricated using Micro Electro Mechanical Systems (MEMS) technology, and then later the MEMS devices including the rigid ring support 41 are aligned and mounted, for example with epoxy resin, onto the end of the elongated body 9. The fabrication is relatively cheap because batch processing is applied.

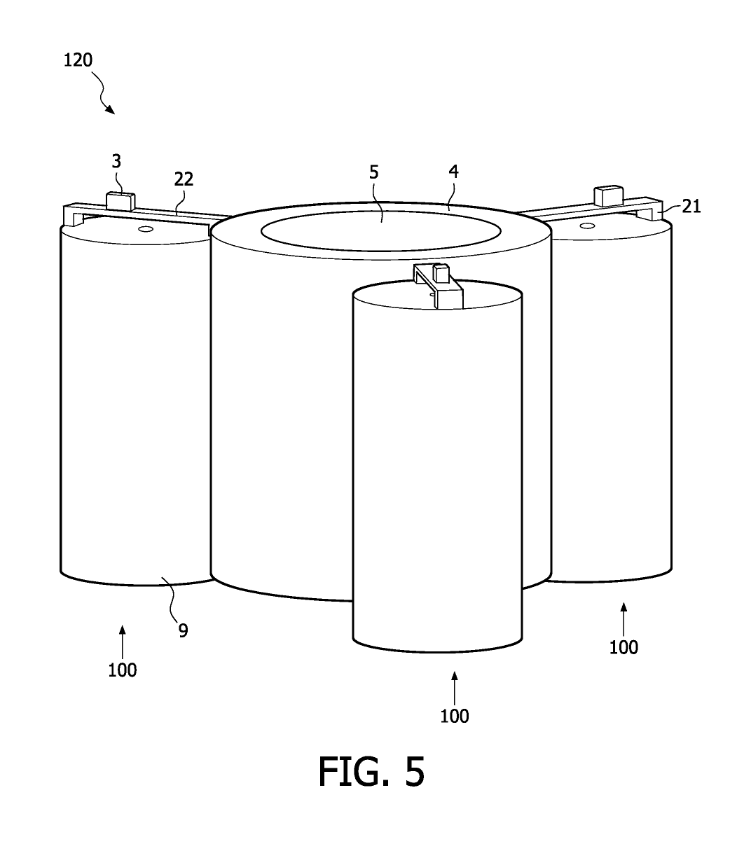

In an embodiment according to the invention the force sensor 100 and/or 110 is combined with a catheter 120 in which the contact force F is detected when the catheter 120 is in contact with a tissue wall, and in which also the spatial orientation with respect to the tissue wall is determined, as is shown in FIG. 5. For this purpose, three force sensors 100 are mounted alongside a fiber 5 surrounded by a cylindrical wall 4. In this case the force sensors 100 are located equidistantly around the perimeter of the catheter 120. The protrusion 3 of each of the force sensors 100 is protruding above the end plane of the catheter 120 such that the protrusion 3 will be the first to contact the tissue wall or surface. By applying three force sensors 100 it is possible to determine the spatial orientation of the catheter 120 with respect to the tissue wall. For this purpose a differential measurement of the voltage or current signals resulting from each contact force F acting on each of the protrusions 3 is performed. Reading out of the position and force can for example be done with a single controller comprising three channels, each having an internal laser source and photodetector respectively.

In this way real-time, instantaneous and quantative information is given to a person operating the catheter 120 on the contact force F at which the catheter 120 is loaded against a target sample, such as for example the tissue wall, and the relative position or spatial orientation of the catheter 120 with respect to the target sample. For example, an approximate perpendicular position of the catheter 120 with respect to the tissue wall results in case each of the three voltage or current signals are kept approximately equal. This avoids misinterpretation of measurements made by the catheter 120 and/or accidental damaging of the tissue due to excess force, for example an oblique position of the catheter 120 or an accidental penetration of tissue. Furthermore, deviations from the perpendicular position can cause a reflection from the tissue surface that can damage the tissue in other parts inside the body. In case the energy dose used for ablation is calculated with respect to the thickness of the tissue, which is known for example by ultrasound imaging, the orientation of the catheter 120 with respect to the tissue surface is important, because a deviation of the catheter from the perpendicular position introduces a change of the thickness of the tissue seen from the catheter 120. If the angle or relative orientation of the catheter 120 with respect to the tissue surface is known, the energy that has to be applied for the ablation can be corrected to a value that compensates for the enlarged thickness of the tissue as seen by the catheter 120. For example in case of an ablation of the pulmonary vein, a protein in the tissue should completely be denaturated throughout the entire tissue thickness. It should be noted that also the embodiment of the force sensor 110 with the flexible disc 42 can be applied in this embodiment.

An example where physical contact of the catheter 120 and the tissue wall has to be monitored during medical treatment is laser ablation of the heart, which requires direct contact of the catheter 120 with the tissue wall to avoid coagulation of red blood cells. In laser ablation it is important to have the part of the catheter 120 that carries out the treatment, in full contact with the tissue wall, in order to avoid blood clotting (due to excessive heat). In this case it is essential to know that the catheter 120 is in appropriate contact with the tissue, as well as to know the contact force F in order to avoid accidental penetration of the tissue due to excess force or improper positioning. An alternative of laser ablation in which the catheter 120 is in full contact with the tissue is to use local irrigation with a fluid, which carries away the red blood cells between the catheter and the treated tissue. In this case the end of the treatment part of the catheter 120, for example an ablation fiber, is on a distance of the tissue, thereby defining a confined space such that a relatively small quantity of the irrigation fluid can carry away the blood from the confined space. This can be achieved passively, by using spacers contacting the tissue, in which it is advantageous to know parameters such as the contact force F between catheter 120 and tissue as well as the spatial orientation of the catheter 120.

The optical power necessary to measure a deflection of the flexible part 22 is in the range of 0.05-0.3 mW, which is low enough not to damage blood cells, and high enough to ensure a good accuracy of the measurement. The diameter of the optical fibers 1 in the elongated cylindrical bodies 9 can be as small as 50 micrometer. The measured contact force F in medical treatment applications is generally in the range of 0.2-1 Newton, which means that the robustness of the force sensor 100,110 is significantly high, resulting in a safe operation.

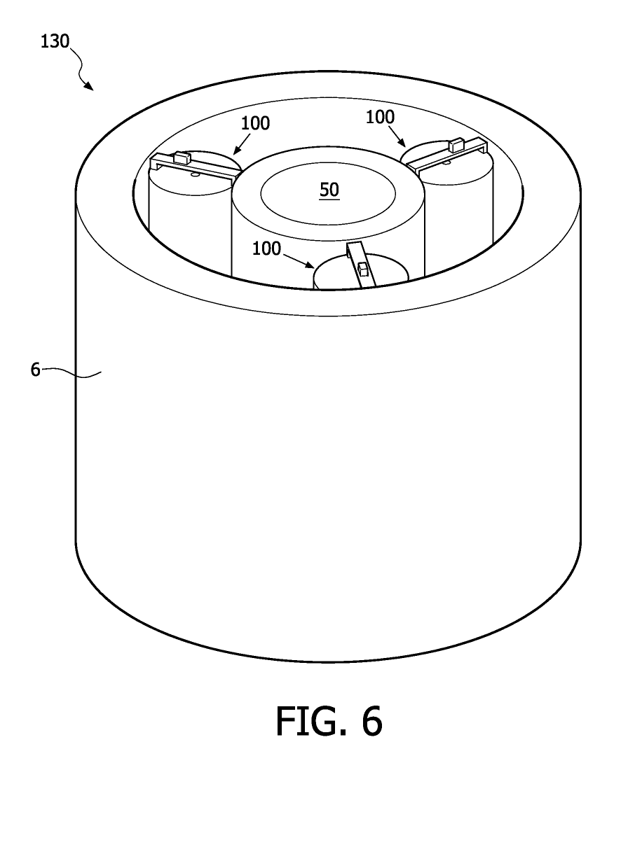

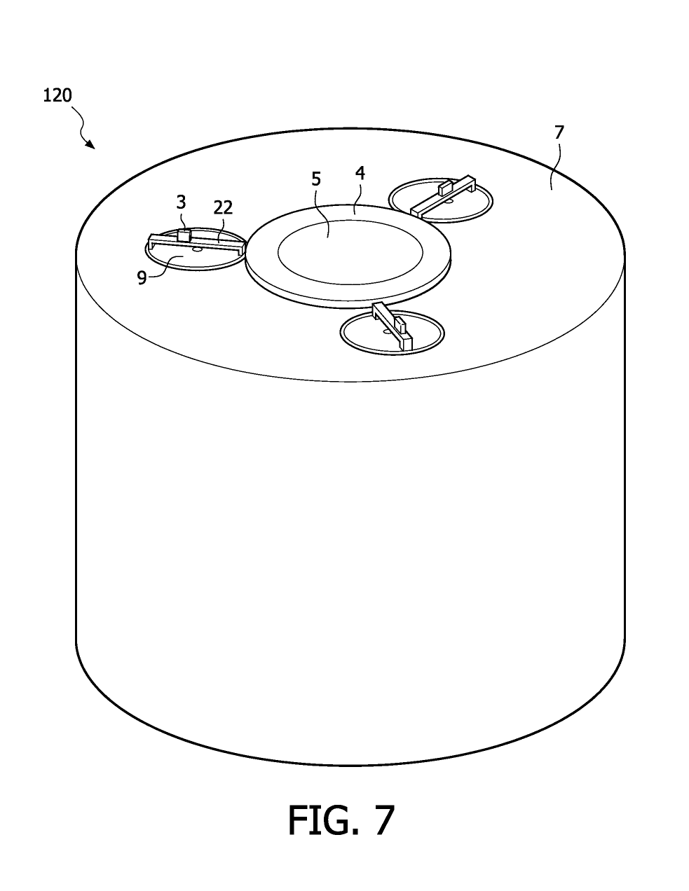

FIG. 6 shows an embodiment of the invention in which the force sensor 100 (and/or the force sensor 110) is integrated in a catheter or endoscope 130 with an ablation fiber 50. Alternatively the catheter or endoscope 130 may be equipped with an electrical wire for RF ablation or a transducer for HIFU (High Intensity Focused Ultrasound) ablation. The three force sensors 100 are positioned at an angle of 120 degrees around the perimeter of the ablation fiber 50. A housing 6 circumscribes the force sensors 100, such that the size of the catheter 130 is confined to minimum. For example, the diameter of the catheter can be as small as 0.30 mm, wherein the diameter of the ablation fiber 50 is 0.10 mm, the diameter of the elongated cylindrical bodies 9 is 0.05 mm, and the thickness of the housing 6 is 0.05 mm. The empty space remaining between the housing 6, the elongated cylindrical bodies 9 and the ablation fiber 50 can serve as a hole or cavity for irrigation liquid flow during ablation for driving away the red blood cells from the ablation path, as well as for cooling of the tissue in case of overheating. Moreover, it gives space for integration of other sensors necessary to monitor the environment during ablation treatment. Such embodiments could comprise a temperature sensor as well as sensors for measurement of electrical signals. In another embodiment the cavities are filled with a filling material 7, for example with epoxy resin (see FIG. 7).

The force sensor 100,110 can be used in combination with other treatment techniques such as RF and High Intensity Focused Ultrasound (HIFU) tissue ablation, because there are no metallic parts required for reading out the signals, and because there is no electrical signal required for the operation of the force sensor 100,110. The force sensor 100,110 can also be embedded in multifiber catheters, for example in basket type catheters. Furthermore, the force sensor 100,110 can be used in applications in which a control of the force F when penetrating tissue on purpose, for example atrial septum, is required. It is also advantageous to apply the force sensor 100,110 for navigation with the catheter 120, 130 towards a target location (e.g. heart) without damaging the walls of the arteries. The force sensor 100,110 will provide a real-time feedback of the contact force F with which the catheter 120,130 touches the artery walls. Furthermore it should be noted that the positioning and contact-force sensing system is MR safe and compatible.

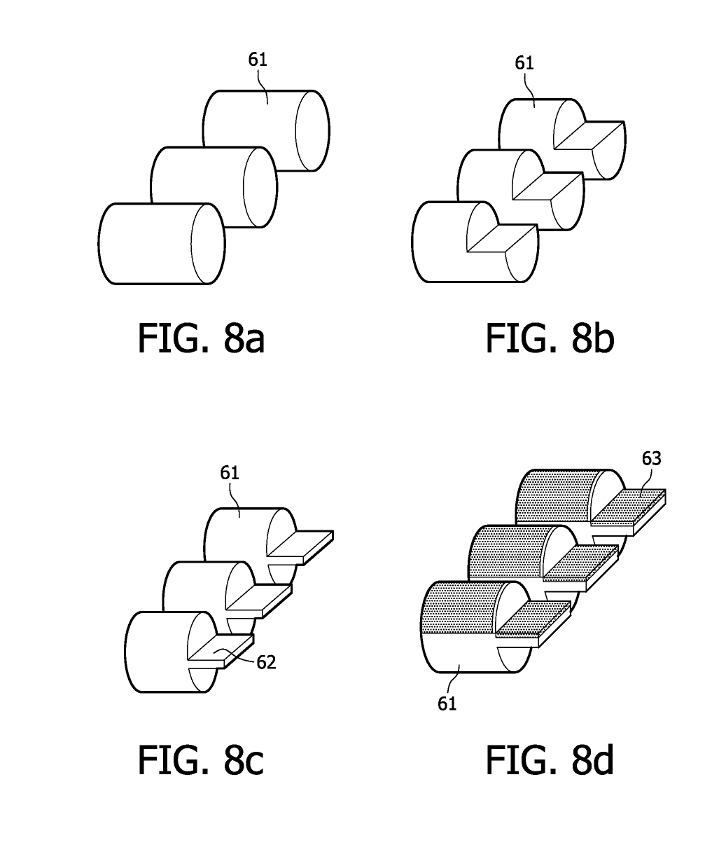

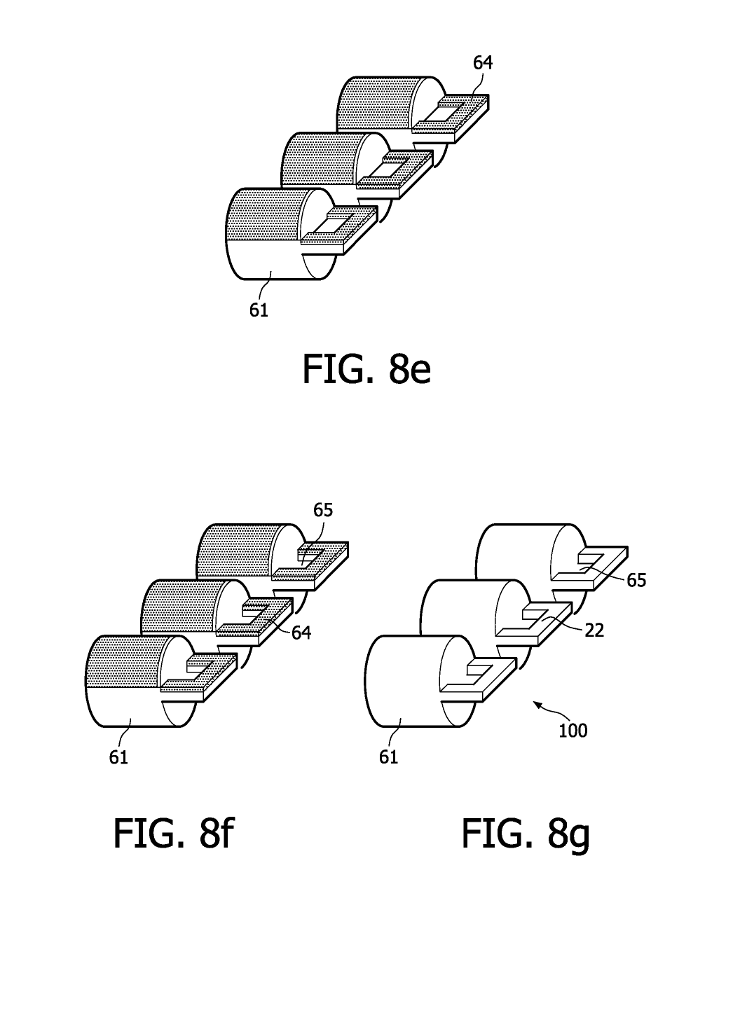

The force sensor 100 can be fabricated by a combination of dicing, photolithography and deep reactive etching processes. An alternative is focused ion beam milling. FIGS. 8a-g illustrate an embodiment of a method of manufacturing the force sensor 100. First cylindrical fibers 61 are mounted and aligned on a first dicing foil (not shown), which is on, but not necessarily attached to, a solid surface, such as a semiconductor wafer. The first dicing foil is, for example, attached to a first annular ring that has an opening of, for example, about 200 mm. Inside the first annular ring, dicing of the cylindrical fibers 61 is performed on the dicing foil, resulting in that a first part of the cylindrical fibers 61 is removed, as is shown in FIG. 8b. It is not required to keep the first dicing foil on the solid surface, because it will be transferred onto a dicing table, which position is very precise with respect to the circular dicing blade, and the annular steel ring locks the first dicing foil into a fixed position. Subsequently, a second dicing foil (not shown) is attached to the opposite side of the cylindrical fibers 61 applying a second annular ring (not shown) to attach the second dicing foil. The first dicing foil, together with the first annular ring, is removed by heating from the cylindrical fibers 61. Then, the second annular ring with the second dicing foil is locked onto the dicing table, and dicing is performed on a side opposite to the removed first part of the cylindrical fibers 61. As a result a second part of the cylindrical fibers 61 is removed, thereby forming a rectangular protruding part 62, as is shown in FIG. 8c. For the subsequent processing steps the cylindrical fibers 61 can stay attached on the second dicing foil. An alternative is to transfer the cylindrical fibers 61 onto a wafer by using a polymer matrix, which at the end of the fabrication process can be dissolved. Next, a mask layer 63 is applied by, for example, sputtering or evaporation of a metal, as is shown in FIG. 8d. A mask pattern 64 is fabricated in the mask layer 63 by, for example, Focused Ion Beam (FIB). Then a, in this case, rectangular opening 65 is formed in the protruding parts 62 by, for example, Reactive Ion Etching (RIE) using the mask pattern 64 to shield the remaining part of the cylindrical fibers 61 from the RIE, as is shown in FIG. 8f. Subsequently the mask pattern 64 is removed, resulting in a part of the force sensors 100 with the bridge-like structures having the flexible part 22, as is shown in FIG. 8g. It should be noted that it is alternatively possible to only apply FIB without the need for the mask pattern 64. The metal mask layer can alternatively be replaced by a photoresist layer in which case the FIB processing is replaced by photolithography.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of other elements or steps than those listed in a claim. The word “a” or “an” preceding an element does not exclude the presence of a plurality of such elements.

1. Medical apparatus (120,130) having a sensor (100,110) for detecting a force acting on the medical apparatus (120,130) in a longitudinal direction, the medical apparatus (120,130) comprising:
an opto-mechanical force transducer having a flexible part (22,42) for receiving the force;
an optical guide (1) having an outcoupling surface (11) that faces the flexible part (22,42) of the opto-mechanical force transducer; and
a photodetector which detects an interference pattern composed of light (32) in the optical guide (1) that is reflected from the outcoupling surface (11) of the optical guide (1) and of light (33) in the optical guide (1) that is reflected from the flexible part (11) of the opto-mechanical force transducer.

2. Medical apparatus (120,130) as claimed in claim 1, in which the flexible part (22,42) of the opto-mechanical force transducer comprises a protrusion (3) that receives the force.

3. Medical apparatus (120,130) as claimed in claim 2, in which the force comprises a contact force (F) between the protrusion (3) and a tissue surface inside a bodily lumen.

4. Medical apparatus (120,130) as claimed in claim 2, in which the protrusion (3) is located outside a path of light that is emitted from the outcoupling surface (11) of the optical guide (1).

5. Medical apparatus (120,130) as claimed in claim 1, in which the medical apparatus (120,130) comprises a closed space region (45) at least confined by the flexible part (42) and the outcoupling surface (11) of the optical guide (1) for detecting a pressure difference between the pressure of the environment and the pressure inside the closed space region (45).

6. Medical apparatus (120,130) as claimed in claim 1, in which the flexible part (22,42) of the opto-mechanical force transducer comprises a bottom surface (23) facing the outcoupling surface (11) of the optical guide (1) and a top surface (24,44) which receives the force and which is opposite to the bottom surface (23), and in which the flexible part (22,42) is supported at its outer side by a rigid supporting part (21,41) mounted on a part of the optical guide (9).

7. Medical apparatus (120,130) as claimed in claim 2, in which the flexible part (22,42) of the opto-mechanical force transducer comprises a flexible bridge connection (22) supported on each side by a rigid bridge support (21) mounted on a part of the optical guide (9) in which the protrusion (3) for receiving the force is located on the top surface (24) of the flexible bridge connection (22).

8. Medical apparatus (120,130) as claimed in claim 6, in which the flexible part (22,42) of the opto-mechanical force transducer comprises a fiber material at least partly coated with a reflective material on the bottom surface (23) or on the top surface (24,44) of the flexible part (22,42).

9. Medical apparatus (120,130) as claimed in claim 1, in which at least three opto-mechanical force transducers (100,110) are mounted around a central elongate part.

10. Medical apparatus (120,130) as claimed in claim 1, further comprising processing logic for computing the force from the interference pattern.

11. Medical apparatus (120,130) as claimed in claim 1, further comprising an optical fiber (50) for laser ablation and a laser source.

12. Method for manufacturing a sensor (100,110) for detecting a force acting on a medical apparatus (120,130) as claimed in claim 7, the method comprising the steps of:
mounting a first side of an optical guide (61) on a first foil;
removing a first part of the optical guide (61);
mounting a second side, which is opposing the first side, of the optical guide (61) on a second foil;
removing the first foil;
removing a second part of the optical guide (61), thereby forming a rectangular protrusion (62) at an end of the optical guide (61);
removing a part of the rectangular protrusion (62) thereby forming a flexible bridge connection (22) having a bottom surface facing an end surface of the optical guide (61) and a top surface opposite to the bottom surface, which flexible bridge connection (22) is supported at its outer side by a rigid bridge support (21), which is attached to the optical guide (61).
